(19) United States
(12) Patent Application Publication
(10) Pub. No.: US 2019/0358300 A1
(43) Pub. Date: Nov. 28, 2019

(54) METHOD AND COMPOSITION FOR NOVEL USE OF IGF-1 AND GARCINOL TO INDUCE CLINICAL WEIGHT LOSS

(71) Applicant: James Ulrick HUGHES, Tampa, FL (US)

(72) Inventor: James Ulrick HUGHES, Tampa, FL (US)

(21) Appl. No.: 16/419,983

(22) Filed: May 22, 2019

Related U.S. Application Data
(60) Provisional application No. 62/674,746, filed on May 22, 2018.

(51) Int. Cl. A61K 38/30; A61K 36/38; A61P 3/04

(52) U.S. Cl. CPC A61K 38/30; A61P 3/04; A61K 36/38

(57) ABSTRACT

Methods and compositions for treating a patient for a condition selected from the group consisting of obesity or diabetes, or a combination thereof, are provided herein. A method includes administering to the patient a compound including a histone acetyltransferase inhibitor (iHAT), or an IgF-1 derivative, and a pharmaceutically acceptable carrier.

### CROSS-REFERENCE TO RELATED APPLICATIONS

This application claims priority to and incorporates by reference the contents of US Provisional Application No. 62/674,746 filed on May 22, 2018.

### BACKGROUND

The gastric mucosa is involved in the regulation of food intake by producing hormones including orexigenic ghrelin and anorexigenic leptin, which act on the hypothalamus (1), (2). Leptin is a 16 kDa protein hormone that plays a key role in regulating energy intake and energy expenditure, including appetite and metabolism. Leptin is an anexorigenic type-1 helical cytokine, an anti-hunger hormone primarily produced and secreted by adipocytes. Leptin is thought to have a central role in basal adipocyte metabolism, control of satiety and obesity. Absence of the mature hormone, leptin, responsible for the obese phenotype of ob/ob mice (3). Ob/ob mice have a mutation in the obese gene, which results in profound obesity, hyperinsulinemia, and hypercorticosteronemia, resulting from a failure to produce mature leptin (3). Mutations in the leptin receptor (LEPR) can have a similar effect, and in mice which suffer from LEPR mutations (db/db mice) a similar phenotype is observed (3). In ob/ob mice, the hypothalamic-pituitary-adrenal axis (HPAA) is activated, and chronic administration of leptin to ob/ob mice decreases plasma corticosterone levels, demonstrating the possibility that the adipose hormone, leptin, is capable of inhibiting the HPAA (3). In humans, obesity does not appear to be a direct result of mutations in the ob gene, or insufficient expression thereof, but instead, satiety and obesity are under more significant, more complex regulation.

At least two populations of leptin-secreting cells exist in the lower half of the gastric mucosa, 1) a population of numerous large cells located around the gastric pits, the Chief epithelial cells (hereinafter “Chief cells”), and 2) a small population of smaller cells scattered between the gastric pits, the endocrine cells. Combined exocrine and endocrine secretions of leptin demonstrate a coordinated role of the gastrointestinal tract in leptin secretion.

Endocrine secretion of leptin and subsequent plasma leptin concentrations decrease dramatically during fasting. The endocrine adipose-hypothalamic axis is generally known to regulate hunger, satiety and obesity. Production of adipokines, cytokines and chemokines by adipose tissue, releases to systemically exert endocrine effects on multiple tissues modulating their physiology. Adipokines influence the hypothalamic-pituitary axis. Among the adipokines, leptin, adiponectin, resistin, chemerin and the peptide kisspeptin, the effect of the leptin on obesity is discussed in detail herein.

Intraluminal concentrations of leptin respond only to distention of the gastric lining, which increases transiently during food intake. Consequently, it has been discovered that exocrine leptin production within the gastrointestinal lumen has an important and predominant role in short-term satiety. Indeed, exocrine leptin has been shown to exert pleiotropic effects on the stomach and intestine. On the stomach, it controls meal size in cooperation with cholecystokinin, stomach emptying, and cytoprotection of the gastric mucosa (4-6). In terms of the exocrine gastric leptin pathway, cells which secrete leptin in this pathway also include LEPR's, in this autocrine-signaling system. Secretion of leptin and subsequent binding of leptin to the same secretory cells creates an amplifying exocrine loop that is intrinsically responsive to short term influences, such as food intake, for example. Leptin provides its function by binding to its receptor (LEPR). Membrane receptors can function to activate intracellular mediators including JAK/STAT pathway, IRS, PI3K. and AMP kinase (7).

Exogenous leptin therapy has shown to be clinically ineffective in the treatment of obesity in humans, likely because alterations of plasma leptin levels result in dramatic changes in compensatory regulation of LEPR concentrations on target tissues.

### BRIEF DESCRIPTION OF THE DRAWINGS

FIG. 1 is a graph displaying data related to the weight of subjects as a percent over target weight during a treatment period.

FIG. 2 includes results of a study showing percent overweight of participants before and after a treatment period, at various time points.

### DETAILED DESCRIPTION

### Overview

IgF-1 (insulin like growth factor) is a small molecular weight protein that potentiates LEPR (leptin receptor) expression on the surface of leptin target cells and downstream leptin receptor-mediated signaling pathways. Insulin like growth factor (IgF-1) is small molecular weight cytokine that competitively binds to cell bound LEPR and prevents down-regulation of LEPR on the cell surface by leptin^{23,24}. Solubilized IgF-1-LEPR complexes also bind to duodenal mucosal cells stimulating the same JAK2 signaling pathways, as does Leptin-LEPR, leading to CCK, PYY and GIP secretion and subsequent satiety. Because of its ability to competitively bind LEPR and prevent down regulation of Leptin secretion by the gastric mucosa, and at the same time form stable IgF-1-LEPR complexes that stimulate duoxlenal secretion of CCK and GIP, enteral IgF1 is an attractive target for therapies used to control obesity. However, enteral IgF1 is extremely labile and survives transit through the gastric lumen only by forming stable solubilized complexes with LepR. The efficacy of IgF-1 is therefore reliant on constituently elevated concentrations of LePR expressed on the surface of gastric Chief cells. The JAK2 signaling pathway is a signaling pathway involved in DNA transcription and expression of genes involved in immunity, proliferation, differentiation, apoptosis and oncogenesis, wherein the JAK2 pathway transmits information from extracellular chemical signals to the nucleus.

HAT (histone acetyltransferase)-modulating compounds that decrease or inhibit the level and/or activity of a HAT may be used for treating or preventing weight gain or obesity in a subject. LEPR and leptin promotor gene transcription are highly regulated by histone acetylation. Histone acetyltransferase inhibitors (iHAT), and specifically an H1 iHat, induce significant long-term increases in both LEPR and leptin gene expression (8-10). Garcinol, a polyisoprenylated benzophenone is a specific H1 iHAT. HAT-modulating compounds that decrease or inhibit the level and/or activity of a HAT may be used, in conjunction with an IgF-1 derivative for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

It has been discovered herein, that presentation of a protein that may competitively bind to a LEPR and prevent binding by free leptin, and consequently preventing downregulation of the JAK2 and possibly other leptin receptor mediated signaling pathways is effective in promoting weight loss in a subject. In one non-limiting example insulin like growth factor (IgF-1) is a small molecular weight protein that competitively binds to LEPR. IgF-1, has been found herein to significantly potentiate LEPR expression on the surface of leptin target cells and downstream leptin receptor mediated JAK2 signaling pathways. In one non-limiting embodiment, a synthetic IgF-1 derivative may be used to induce the effects described herein related to IgF-1.

LEPR and leptin promotor gene transcription are highly regulated by histone acetylation. Histone acetyltransferase inhibitors (iHAT, more specifically, H1 iHAT, in one non-limiting embodiment) induce significant increases in both LEPR and leptin gene expression. Epigenetic modifications of the leptin promoter have been linked with leptin gene transcription. Typically, the acetylation of histones H3 and H4 by histone acetyltransferases (HATs) induces an open chromatin conformation in DNA that allows the transcription machinery to access the promoter, leading to the activation of gene expression. This is a mechanism by which leptin gene transcription can be enhanced. In a novel combination, first described herein, a synthetic IgF-1 derivative, augmented with an H1 iHAT provides a synergistic result in achieving weight loss in obese or overweight patients. The weight loss achieved has found to be both clinically significant and sustained. In one non-limiting embodiment, the H1 iHAT may include garcinol, a polyisoprenylated benzophenone.

Additionally, or alternatively, HAT-modulating compounds, specifically iHAT compounds such as H1 iHAT, that decrease the level and/or activity of a HAT may be used for reducing appetite and/or increasing satiety, thereby inducing weight loss or preventing or reducing weight gain. In combination with IgF-1 or an IgF-1 derivative, a combination treatment of IgF-1 and iHAT provides an unexpected synergistic effect in treating obesity, inducing weight loss, or preventing or reducing weight gain. A patient in need of such a treatment may be a one who is overweight, obese or predisposed to obesity, or to becoming overweight. The method may comprise administering a composition comprising a combination of an iHAT, such as garcinol, for example, and an IgF-1 to the patient according to a dosage regimen. In some embodiments, dosage may be provided in cc/kg of body weight. For example, a subject may receive between 0.05 and 1.5 cc/kg body weight of the composition one or more times per day. In other embodiments, a subject may receive between 0.1 cc/kg body weight and 0.5 cc/kg body weight at least once per day. In one particular, non-limiting embodiment, the subject may receive 0.2 cc/kg per body weight of the composition, administered twice per day.

In another non-limiting embodiment, a compound comprising an iHAT, such as garcinol, for example, may be administered to a patient in need thereof, in conjunction with a compound comprising IgF-1. This combination is particularly effective in reducing weight gain and/or increasing weight loss over time in a patient. This combination is also particularly effective in achieving satiety in a patient, and/or reducing appetite in the patient, and sustaining weight loss.

In still a further embodiment, a method of treating a patient for a condition, selected from the group consisting of: obesity, diabetes, and atopic dermatitis comprises co-administering to the patient a compound or compounds comprising: 1) an iHAT, and 2) an IgF-1 or IgF-1 derivative. In yet a further embodiment, the method comprises co-administering a compound including iHAT, a compound including IgF-1 or an IgF-1 derivative, and a pharmaceutically acceptable carrier.

In a further embodiment, A composition comprising a histone acetyltransferase inhibitor (iHAT), and an IgF-1 derivative is provided. In yet a further embodiment, the composition may include pharmaceutically acceptable carrier.

In still a further embodiment, a method of increasing leptin receptor and leptin gene expression in a patient, comprising, administering to the patient a compound comprising an effective amount of an iHAT and/or an effective amount of an IgF-1 derivative. In one non-limiting embodiment the iHAT comprises an H1 iHAT. In still a further embodiment, the H1 iHAT may include garcinol, a polyisoprenylated benzophenone. In a further embodiment, the IgF-1 may specifically include a synthetic IgF-1 derivative.

### EXAMPLES

Oral IgF-1 Augmented with Garcinol Results in Clinically Significant and Sustainable Weight Loss in a Random Population of Overweight and Obese Pet Canines

In a clinical study of canine pets, subjects were selected randomly from routine clinically overweight or obese subjects seen for routine medical health care. Subject suffering from acute medical disease were excluded from the study, however subjects with comorbidities, that were well managed, ie diabetes, heart disease, thyroid or adrenal disease, were not excluded. Subjects were deemed overweight if they received a score of >3.0 but <4.0 on the AVCIM body score index, and obese with a score of >4.0. No placebo group was included in the study, as numerous historical studies on the effect of diet on canine obesity have been published. Subjects received full blood work panels at the start of the study, including endocrine profiling. Subjects with endocrine disease were treated concomitantly during the study when identified as a new case. Those subjects currently being treated for medical conditions that did not preclude them from the study continued to receive appropriate treatment. The medical conditions of subjects were monitored during the course of the study. All subjects in the study received the same dose per body weight of the medication, administered at the same schedule. The medication was dosed to the subjects at 0.2 cc/kg body weight given twice daily. The study group was randomly divided into subjects receiving biweekly cyanocobalamin (Vit B₁₂) injections at dose of 0.3 mg/kg intramuscularly and those that did not receive biweekly cyanocobalamine injections. Owners were recommended to change to feeding a good quality non-prescription grain-free diet, and given a recommendation as to quantity to feed, however, no attempt was made to oversee the feeding and additional caloric supplementation of the animals in this study. Subject weights were revaluated biweekly for a period of 90 days. The target weight for subjects was determined by AKC breed standards. Where the breed of a subject was indeterminant, the target weight was determined by extrapolating from the AVCIM BSI score as well as the clinical acumen of the clinician. Follow up was performed on a number of subjects 1 year following the study.

### Materials and Methods

Human IgF1-70^{a} was reconstituted to a concentration of 100 μg/ml in PBS. The stock solution of IgF was stored at −20 C. and used for the source of the oral medication. A stock solution of Garcinol was prepared by solubilizing Garcinol^{b} in 90% EtOH @ 32′C to a final concentration of 2 mg/ml and storing at 27′C. The oral medication was prepared by adding 20 μg of IgF and 1 mg of Garcinol to 2 cc of a propriety blend of salmon oil, polyethylene glycol, glycerin and buffered Tris solution. The solution was indirectly sonicated 200 W @ 20 kHz for 5 minutes in an ice water bath. Observing clarification of the opaque suspensions was deemed a test of successful miscellation of suspensions. Multilaminar micelles and liposomes were observed by direct microscopic examination. No attempt was made control the size, density or multilaminar structure of the micelles. The suspension was then added to an inert polysaccharide solution with added flavoring just prior to dispensing to a final concentration of 2 μg/ml of IgF and 100 μg/ml of Garcinol.

### Results

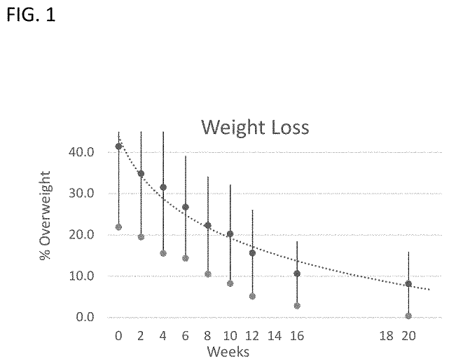

A random sample of 83 canine subjects were included in the study population. The average age of subjects in the study was 8.6 years with 62% of subjects were >8 years old. The sex of subjects was almost evenly matched (females=57%, males=43%), and the vast majority of subjects were altered (81%) consistent with the greater population of pet animals in urban areas. Subjects having recorded comorbidities comprised 38% of the group. The average AVCIM BSI values for subjects was 3.64 with 74% of subjects having a score >3.6. The weights of subjects expressed as a percentage above approximated normal weight during treatment is displayed (FIG. 1.). FIG. 1 shows the weight of subjects expressed as a percent over target weight during a twenty (20) week treatment period.

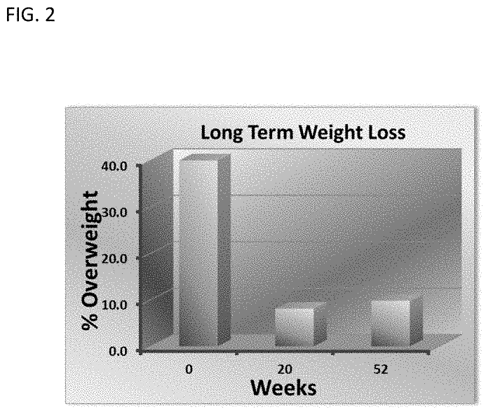

As shown in FIG. 2, after 52 weeks following the study, results of a subset of the study population group (33 subjects) are indicated below. This population was seen for routine healthcare needs and management of concomitant medical conditions, however none of the subjects in this subgroup received additional medication or dietary guidance. As provided in the chart shown in FIG. 2, the subjects were within the 40% overweight category, upon completion of the study at 20 weeks, these subjects were below 10% overweight. At 52 weeks post initiation of the study, the overweight percentage of these subjects had slightly increased, but remained under 10% overweight, demonstrating the sustained weight loss results of treatment with IgF and iHAT.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### Definitions

The term “an effective amount” means an amount of an agonist or antagonist satiety factor or compositions comprising thereof that when, administered to a subject for treating a disease is sufficient to effect such treatment for the disease. An effective amount can vary depending on, inter alia, the satiety factor used or the particular combination of compounds used, the disease and its severity and the age, weight, etc. of the subject to be treated. In a further embodiment, an effective amount may include an amount sufficient to induce clinically significant and sustained weight loss in obese or overweight patients.

“Preventing” or “prevention” refers to a reduction in the risk of acquiring a disease or disorder (i.e., causing at least one of the clinical symptoms of the disease not to develop in a subject that may be exposed to or predisposed to the disease but does not yet experience or display symptoms of the disease). Preferably, prevention refers to the use of a compound or composition in a subject not yet affected by the disease or disorder or not yet exhibiting a symptom of the disease or disorder, for instance a subject not yet infected or not yet exhibiting the symptoms of infection.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the term “administering” or “administration” includes but is not limited to oral or intravenous administration by liquid, capsule, tablet, or spray. Administration may be by injection, whether intramuscular, intravenous, intraperitoneal or by any parenteral route. Parenteral administration can be by bolus injection or by continuous infusion. Formulations for injection may be presented in unit dosage form, for example, in ampoules or in multi-dose containers with an added preservative. The compositions may take the form of suspensions, solutions or emulsions in oily or aqueous vehicles and may contain formulatory agents such as suspending, stabilizing and/or dispersing agents. Alternatively, the compositions may be in powder form (e.g., lyophilized) for constitution with a suitable vehicle, for example sterile pyrogen-free water, before use. Compositions may be delivered to a female subject by inhalation by any presently known suitable technique including a pressurized aerosol spray, where the dosage unit may be controlled using a valve to deliver a metered amount.

The term “in conjunction with” as used herein includes but is not limited to synchronously or near synchronous timing, the phrase may also include the timing of outputs, where one output directly follows another output.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

As used herein, the term “IGF-1 derivative” includes IGF-1 (insulin like growth factor 1) and its derivatives, for example, it may include natural or synthetic IGF-1, or LR3 IGF-1 (long chain variant of IGF-1) as a variant, or derivatives thereof.

Administration by capsule and cartridges containing powder mix of the composition can be used in an inhaler or insufflator to deliver the particles to the female subject. Still other routes of administration which may be used include buccal, urethral, vaginal, or rectal administration, topical administration in a cream, lotion, salve, emulsion, or other fluid may also be used.

The term “co-administration” or “co-administering” as used herein refer to the administration of a substance before, concurrently, or after the administration of another substance such that the biological effects of either substance synergistically overlap.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced items. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein.

### Dosage

The dose administered to an animal, particularly a human, in accordance with the present invention should be sufficient to effect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

The amount of the compound of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will insure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

### Pharmaceutical Compositions

Various embodiments of the invention are foreseen to have valuable application as constituents of pharmaceutical preparations to treat various conditions generally defined as pathologies. Accordingly, embodiments of the invention also comprise pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. In some embodiments, dosage may be provided in cc/kg of body weight. For example, a subject may receive between 0.05 and 1.5 cc/kg body weight of the composition one or more times per day. In other embodiments, a subject may receive between 0.1 cc/kg body weight and 0.5 cc/kg body weight at least once per day. In some embodiments, the dosage may increase to twice per day, or three times per day or more. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol and cellulose acetate. The compositions may be contained in a vial, sponge, syringe, tube, or other suitable container.

A pharmaceutical formulation may be provided for oral or parenteral administration, in which case the formulation may contain the compounds and/or compositions described herein, but may contain alternative pharmaceutically acceptable carriers, vehicles, additives, etc. particularly suited to oral or parenteral drug administration. Alternatively, the compounds and/or compositions described herein having the active ingredients, an H1 iHAT (e.g. garcinol) and IgF-1, or a derivative thereof, may be administered orally or parenterally substantially as described above, without modification.

One possibility to achieve sustained release kinetics is embedding or encapsulating the active compound into nanoparticles. Nanoparticles can be administrated as powder, as a powder mixture with added excipients or as suspensions. Colloidal suspensions of nanoparticles can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. In certain embodiments, nanoparticles are preferred having a 5 diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out, mechanical grinding, microprecipitation, and by emulsification diffusion. With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently.

Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986. Further description on preparing nanoparticles can be found, for example, in U.S. Pat. No. 6,264,922, the contents of which are incorporated herein by reference. Liposomes are a further drug delivery system which is easily injectable.

Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent; for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

### REFERENCES

1. Bado A. Levasseur S, Attoub S. Kermorgant S, Laigneau J P. Bortoluzzi M N, Moizo L. et al. (1998) The stomach is a source of lceptin. Nature 394:790-793.
2. Wynne K. Stanley S. Bloom S (2004) The gut and regulation of body weight. J Clin Endocrinol Metab 89:2576-2582.
3. Heiman, M., Ahima, R. Craft, L. Schoner, B. Stephens, T., Flier, J. (1997) Leptin Inhibition of the Hypothalamic-Pituitary-Adrenal Axis in Response to Stress. Endocrinology. Vol. 138, No. 9.
4. Brzozowski T, Konturek P C, Konturek S J, Pajdo R, Duda A, Pierzchalski P, Bielanski W, et al. (1999) Leptin in gastroprotection induced by cholecystokinin or by a meal. Role of vagal and sensory nerves and nitric oxide. Eur J Pharmacol 374:263-276.
5. Attoub S, Noe V, Pirola L, Bruyneel E, Chastre E, Mareel M, Wymann M P, et al. (2000) Leptin promotes invasiveness of kidney and colonic epithelial cells via phosphoinositide 3-kinase-, rho-, and rac-dependent signaling pathways. FASEB J 14:2329-2338.
6. Barbier M, Attoub S, Galmiche J P. Leptin: physiological aspects and implications in hepato-gastroenterology (2000) Gastroenterol Clin Biol 24:506-519.
7. Bjorbaek C, Kahn B B (2004) Leptin signaling in the central nervous system and the periphery. Recent Prog Horm Res 59:305-331.
8. Wenwen Shen, et. al; “Epigenetic Modification of the Leptin Promoter in Diet-Induced Obese Mice and the Effects of N-3 Polyunsaturated Fatty Acids”; Sci Rep. 2014; 4: 5282.
9. Crujeiras A B, et al.; “Leptin resistance in obesity: An epigenetic landscape”; Life Sci.: 2015; November 1; 140:57-63.
10. Tho X. Pham and Ji-Young Lee; “Epigenetic Regulation of Adipokines”; Int J Mol Sci.: 2017; August; 18(8): 1740.

1. A method of treating a patient for a condition, selected from the group consisting of: obesity or diabetes, or a combination thereof, comprising:
administering to the patient a compound comprising:
a histone acetyltransferase inhibitor (iHAT), or
an IgF-1 derivative; and
a pharmaceutically acceptable carrier.

2. The method of claim 1, wherein the iHAT and the IgF-1 derivative are co-administered to the patient.

3. The method of claim 1, further comprising co-administering the iHAT and the IgF-1 derivative with one or more anti-obesity agents.

4-9. (canceled)

10. A method of increasing leptin receptor and leptin gene expression in a patient, comprising, administering to the patient a compound comprising an effective amount of an iHAT and/or an effective amount of an IgF-1 derivative.

11. The method of claim 10, wherein the iHAT comprises an H1 iHAT.

12. The method of claim 11, wherein the H1 iHAT comprises a polyisoprenylated benzophene.

13. The method of claim 12, wherein the polyisoprenylated benzophene comprises garcinol.

14. The method of claim 10, wherein the IgF-1 derivative binds to LEPR in the patient.

15. A composition comprising:
a histone acetyltransferase inhibitor (iHAT), and
an IgF-1 derivative.

16. The composition of claim 15, further comprising a pharmaceutically acceptable carrier.
